(19) United States
(12) Patent Application Publication
(10) Pub. No.: US 2023/0075134 A1
(43) Pub. Date: Mar. 9, 2023

(54) METHOD FOR ISOLATING AND PURIFYING MESENCHYMAL STEM CELLS FROM HERNIA SAC AND METHOD FOR TISSUE REPAIR BY USING MESENCHYMAL STEM CELLS FROM HERNIA SAC

(71) Applicant: Joshua Medical Holdings Co. Ltd., Taipei City (TW)

(72) Inventor: Dian-Yu Lin, Taipei (TW)

(21) Appl. No.: 17/895,418

(22) Filed: Aug. 25, 2022

Related U.S. Application Data
(60) Provisional application No. 63/238,800, filed on Aug. 31, 2021.

(51) Int. Cl. A61K 35/28; C12N 5/0775; A61K 45/06

(52) U.S. Cl. CPC A61K 35/28; C12N 5/0668; A61K 45/06; C12N 2500/32

(57) ABSTRACT

The present disclosure provides a method for isolating and purifying mesenchymal stem cells from hernia sac and a method for tissue repair by using the mesenchymal stem cells from hernia sac. The mesenchymal stem cells isolated and purified by the method of the present disclosure have high yield, high cell division ability, do not need to use enzymes as necessary reagents, and have high potential for application in regenerative medicine or tissue engineering for tissue repair.

### CROSS-REFERENCE TO RELATED APPLICATION

This application claims priority of U.S. Provisional Application No. 63/238,800, filed on Aug. 31, 2021, the content of which is incorporated herein in its entirety by reference.

### BACKGROUND OF THE INVENTION

### 1. Field of the Invention

The present invention relates to a method for isolating and purifying mesenchymal stem cells from hernia sac and a method for tissue repair by using the mesenchymal stem cells from hernia sac.

### 2. The Prior Art

So far, mesenchymal stem cells have been clinically proven to be a kind of valuable stem cells, which have the functions of immune modulation, helping to repair tissues and so on. In addition, some experts have publicly pointed out that the number of mesenchymal stem cells with the above-mentioned functions is related to the degree of function exertion. The more the number of cells, the higher the degree of function exertion; the less the number of cells, the lower the degree of function exertion. Sources of mesenchymal stem cells include embryonic stem cells and adult stem cells, among which adult stem cells exist in bone marrow, umbilical cord, placenta, blood vessel wall and subcutaneous fat, etc. Those skilled in the art all use similar culture technology to expand and culture, which can be made into biological products. However, in the prior art, the steps for obtaining adult stem cells are too cumbersome, affecting the quality and inconsistency, and it is also impossible to effectively screen and obtain a large number of primary cultured (P0) adult stem cells.

Since mesenchymal stem cells have the advantages of immune modulation, helping to repair tissues in order to achieve the above-mentioned proven effective functions, the required number of cells would be one of the key factors. Those skilled in the art usually need to culture the obtained stem cells in vitro for several passages in order to achieve the purpose of increasing the number of cells. At present, there are products advertised as mainly placental mesenchymal stem cells on the market, but some buyers worry that the product is produced by using animal serum-containing culture medium and mixed whole placental tissue. Because buyers are worried that if the ingredients and sources of the product are too mixed, it is easy to reduce the willingness to buy the product.

Due to the complex sources of existing cells to obtain, it is difficult to ensure product quality, including that it must be able to produce cells that are exactly the same as the original cells under long-term expansion and culture. In addition, traditional mesenchymal stem cell isolation and purification methods often use enzymes (such as collagenase) as necessary reagents. However, the mesenchymal stem cells obtained by the traditional method of separation and purification of mesenchymal stem cells using collagenase often have the disadvantages of low yield and poor properties of mesenchymal stem cells (for example, insufficient cell division ability).

In order to solve the above-mentioned problems, those skilled in the art urgently need to develop a novel method for isolating and purifying mesenchymal stem cells for the benefit of a large group of people in need thereof.

### SUMMARY OF THE INVENTION

An objective of the present invention is to provide a method for isolating and purifying mesenchymal stem cells from hernia sac, comprising the following steps: (a) obtaining a hernia sac specimen and cutting the hernia sac specimen into tissue pieces; (b) washing the tissue pieces and standing for a first predetermined time to form a supernatant and washed tissue pieces, then removing the supernatant; (c) adding a first medium to the washed tissue pieces and performing a homogenization treatment, followed by a resuspension treatment to form a mixture comprising the mesenchymal stem cells, filtering the mixture comprising the mesenchymal stem cells to obtain a filtrate, and inoculating the filtrate into a flask for culturing for a second predetermined time; and (d) separating the mesenchymal stem cells attached on the flask, then adding a second medium and continuing to culture for a third predetermined time, thereby purifying the mesenchymal stem cells; wherein the step (b) is performed without the addition of an enzyme.

Another objective of the present invention is to provide a method for tissue repair, comprising administering to a subject in need thereof a pharmaceutical composition comprising an effective amount of mesenchymal stem cells from hernia sac, wherein the mesenchymal stem cells from hernia sac are isolated and purified by the abovementioned method.

According to an embodiment of the present invention, the tissue repair is muscle tissue repair.

According to an embodiment of the present invention, the tissue pieces have a volume of 1-2 mm³.

According to an embodiment of the present invention, the first predetermined time is 4-6 minutes.

According to an embodiment of the present invention, step (b) is repeated no more than twice.

According to an embodiment of the present invention, both the first medium and the second medium are alpha-minimal essential medium.

According to an embodiment of the present invention, the first medium or the second medium is further supplemented with 10% fetal bovine serum (FBS) and 1% solution comprising penicillin and streptomycin.

According to an embodiment of the present invention, the second predetermined time is 24-48 hours.

According to an embodiment of the present invention, in step (d), the mesenchymal stem cells attached on the flask are separated by trypsin.

According to an embodiment of the present invention, the third predetermined time is 7-10 days.

According to an embodiment of the present invention, the hernia sac is from an inguen.

In summary, the method for isolating and purifying mesenchymal stem cells from hernia sac and the method for tissue repair by using the mesenchymal stem cells from hernia sac of the present invention have the following effect. The mesenchymal stem cells of the present invention have high yield, high cell division ability, do not need to use enzymes as necessary reagents, and have high potential for application in regenerative medicine or tissue engineering for tissue repair (such as muscle tissue repair) that can be applied to the patient's own hernia repair defect structure.

### BRIEF DESCRIPTION OF THE DRAWINGS

The following drawings form part of the present specification and are included here to further demonstrate some aspects of the present invention, which can be better understood by reference to one or more of these drawings, in combination with the detailed description of the embodiments presented herein.

FIG. 1 shows the hernia sac during inguinal hernia surgery.

FIG. 2 shows the cumulative cell number of three different methods for hernia mesenchymal stem cell (MSC) cultivation.

FIG. 3 shows plastic adherence properties and morphology of cultured hernia sac-derived cells.

FIG. 4 shows positive and negative markers used to identify the presence of mesenchymal stem cells in the hernia sac, wherein MSC represents mesenchymal stem cell, and SAC represents hernia sac.

FIG. 5 shows differentiation ability and identification of mesenchymal stem cells following von Kossa, Alcian blue, and Oil Red O staining.

FIG. 6 shows that the hernia sac derived cells have more mesenchymal stem cells than the subcutaneous fat derived cells, wherein CFU represents colony forming unit, and * represents p<0.05.

FIG. 7 shows that the hernia sac derived mesenchymal stem cells have a higher number of cell divisions and more accumulated cells than the subcutaneous fat derived mesenchymal stem cells.

### DETAILED DESCRIPTION OF THE PREFERRED EMBODIMENT

In the following detailed description of the embodiments of the present invention, reference is made to the accompanying drawings, which are shown to illustrate the specific embodiments in which the present disclosure may be practiced. These embodiments are provided to enable those skilled in the art to practice the present disclosure. It is understood that other embodiments may be used and that changes can be made to the embodiments without departing from the scope of the present invention. The following description is therefore not to be considered as limiting the scope of the present invention.

### Definition

As used herein, the data provided represent experimental values that can vary within a range of ±20%, preferably within ±10%, and most preferably within ±5%.

Unless otherwise specified in the context, the terms “a”, “the” and similar terms used in the specification (especially in claims described later) should be understood to include both the singular and the plural forms.

The medicament provided according to the present invention can be in any suitable form without special limitation, and it is in a corresponding suitable dosage form depending on the intended use. For example, but not limited to, the medicament can be administered to a subject in need by non-oral (e.g., transdermal) administration.

According to the present invention, the medicament can be manufactured to a dosage form suitable for parenteral administration, using techniques well known to those skilled in the art, including, but not limited to, injection (e.g., sterile aqueous solution or dispersion), sterile powder, dispersible powder or granule, solution, suspension, emulsion, and the like.

The medicament according to the present invention may be administered by a parenteral route selected from the group consisting of: intraperitoneal injection, subcutaneous injection, intraepidermal injection, intradermal injection, intramuscular injection, intravenous injection, intralesional injection, sublingual administration, and transdermal administration.

According to the present invention, the medicament may further comprise a pharmaceutically acceptable carrier which is widely used in pharmaceutically manufacturing techniques. For example, the pharmaceutically acceptable carrier can comprise one or more reagents selected from the group consisting of solvent, emulsifier, suspending agent, decomposer, binding agent, excipient, stabilizing agent, chelating agent, diluent, gelling agent, preservative, lubricant, absorption delaying agent, liposome, and the like. The selection and quantity of these reagents fall within the scope of the professional literacy and routine techniques of those skilled in the art.

According to the present invention, the pharmaceutically acceptable carrier comprises a solvent selected from the group consisting of water, normal saline, phosphate buffered saline (PBS), sugar-containing solution, aqueous solution containing alcohol, and combinations thereof.

Taking the dosage form suitable for transdermal administration as an example, the medicament provided according to the present invention can be in the form of patches, lotions, creams, gels (for example: hydrogels), pastes (for example: dispersion paste, ointment), spray, or solution (for example: suspension), etc., but not limited to this.

The medicament provided according to the present invention can be administered with different administration frequencies such as once a day, multiple times a day, or once a few days, depending on the individual's needs, age, weight, and health status. In the medicament provided according to the present invention, the content ratio of mesenchymal stem cells in the medicament can be adjusted according to actual application requirements. In addition, the medicament may additionally contain one or more other active ingredients (e.g., tissue repair drugs) as needed, or be used in combination with a drug containing the one or more other active ingredients to further enhance the efficacy of the drug or increase the flexibility and compounding degree of the formulation, as long as the other active ingredients are effective against the active ingredients of the present invention (that is, inguinal hernia sac derived mesenchymal stem cells) without adverse effects.

The present invention is further illustrated by the following examples. The embodiments are provided for illustration only, but not for limiting the scope of the present invention. The scope of the present invention is shown in the appended claims.

### Example 1

Method for Isolating and Purifying Mesenchymal Stem Cells from Inguinal Hernia Sacs

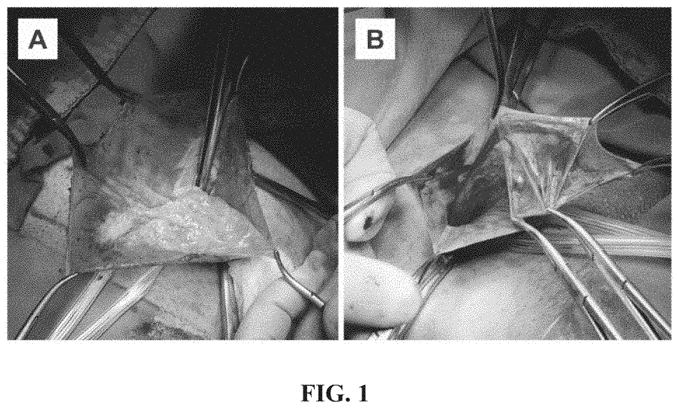

Surgery for adult inguinal hernia (see FIGS. 1A and B both represent the hernia sac during surgery) involves performing the open method with inguinal incision. Fresh hernia sacs obtained from 4 randomly selected inguinal hernia surgeries were harvested and immediately sent to the laboratory. Details regarding these 4 patients are summarized in Table 1. Mesenchymal stem cells (MSCs) were harvested within 48 h. Peritoneal tissue from hernia sac (i.e., hernia sac specimen) was first isolated, and about 2 g of hernia sac specimen was cut into 1-2 mm³ tissue pieces by scalpel. The tissue pieces were washed by 10 ml phosphate buffered saline (PBS), standing for about 4-6 min to form a supernatant and washed tissue pieces, followed by removing the supernatant. This step was repeated no more than twice. The mesenchymal cells were obtained using the Miltenyi gentleMACS Dissociator, which has been used for the semiautomated dissociation of tissues into single-cell suspensions or thorough homogenates. The dissociated suspension was filtered through 40-μm strainer (BD Falcon) and cultured in alpha-Minimal Essential Medium (Thermofisher) plus 10% fetal bovine serum (FBS) (Hyclone) at 37° C., 5% CO₂ and humidified atmosphere. Alternatively, after the above step which was repeated twice, the washed tissue pieces were resuspended in 2 ml alpha-Minimal Essential Medium supplemented with 10% fetal bovine serum (FBS) and 1% solution comprising penicillin and streptomycin to form a mixture comprising the mesenchymal stem cells, followed by seeding the mixture comprising the mesenchymal stem cells into a 25 cm² flask and culturing for 24-48 hours. The mesenchymal stem cells attached on the flask were separated, and 3 ml of medium was added and continued to culture for 7-10 days, thereby purifying the mesenchymal stem cells. In particular, cells appear to grow out of tissue pieces on 3-4 days. When cells grow to about 60% confluency of flask, trypnised to detach the cells and tissue pieces, then passing through a 40 μm strainer. This experiment had been reviewed and approved by institutional review board of Tung's Taichung Metro Harbor Hospital. All patients and participants provided written informed consent prior to participation (IRB #109060).

TABLE 1

| | Case 1 | Case 2 | Case 3 | Case 4 |
|---|---|---|---|---|
| Age | 54 | 36 | 71 | 42 |
| Sex | Male | Male | Male | Male |
| Height | 165 | 166 | 156 | 174 |
| Weight | 70 | 77 | 56.2 | 87 |
| Smoking | No | No | No | No |
| Hernia | 32 × 14.5 × | 6 × 4 × | 2 × 3 × | 3 × 3 × |
| size | 2.5 cm | 8 cm | 5 cm | 5 cm |
| Medical | Hepatitis B, | No | Prostate | Lung |
| history | hypertension | | cancer | arteriovenous |
| | | | | aneurysm, |
| | | | | scrotal |
| | | | | spermatic |
| | | | | varicocele |
| Surgical | No | No | Robot-assisted | Embolization |
| history | | | laparoscopic | |
| | | | radical | |
| | | | prostatectomy | |

The above is the method flow of the present invention for separating and purifying mesenchymal stem cells from hernia sac, also known as direct culture, without the use of enzymes to separate tissues, hereinafter referred to as method A. The conventional methods are used as a comparative group: method B and method C, both of which require enzymes to separate tissues to obtain single cells. Method B is the method used in general papers, and its procedure is as follows. Peritoneal tissue from hernia sac (i.e., hernia sac specimen) was first isolated, and about 2 g of inguinal hernia sac specimen was cut into 2-4 mm³ tissue pieces by scalpel. 5 ml collagenase (1 mg/ml) solution was added and incubated at 37° C., 2 hr, followed by filtrating with 100 μm strainer to remove tissue pieces. Centrifugation was performed at 200×g, 10 min and the supernatant was removed, followed by adding 5 ml alpha-Minimal Essential Medium supplemented with 10% fetal bovine serum (FBS) and 1% solution comprising penicillin and streptomycin to the cell pellet and transferring into a 25 cm² flask. The main difference between the present invention (method A) and the conventional method (method B) is that: the tissue pieces of method A do not need to be treated with enzymes, such as collagenase; the tissue pieces of method B need to be treated with collagenase. The source of collagenase is bacterial production, so there is a problem of purity, and there are more likely residual bacterial endotoxins. In addition, the strains that produce this enzyme are generally pathogenic bacteria. So it is best not to use it as much as possible. However, there are few unnecessary ones. The quality and quantity of the source of the hernia sac derived mesenchymal stem cells of the present invention are strong, so collagenase may not be used. In the method A of the present invention, the mesenchymal stem cells can be obtained by crushing into a homogenate with a dissociator, thereby replacing the method of enzymatic decomposition. It means that the hernia sac derived mesenchymal stem cells can obtain the results of strong differentiation, high yield, and high division ability by using method A.

The procedure of method C is as follows. Peritoneal tissue from hernia sac (i.e., hernia sac specimen) was first isolated, and about 2 g of inguinal hernia sac specimen was cut into 2-4 mm³ tissue pieces by scalpel. 5 ml collagenase (1 mg/ml) solution was added and the gentleMACS dissociator was used for 20 min to get single cell solution, followed by filtrating with 40 μm strainer to remove tissue pieces. Centrifugation was performed at 200×g, 10 min and the supernatant was removed, followed by adding 5 ml alpha-Minimal Essential Medium supplemented with 10% fetal bovine serum (FBS) and 1% solution comprising penicillin and streptomycin to the cell pellet and transferring into a 25 cm² flask. In particular, the initial cell culture flasks of methods B and C contain no tissue pieces, but lots of blood cells. The main difference between the present invention (method A) and the conventional method (method C) is that: the tissue pieces of method A do not need to be treated with enzymes, such as collagenase; the tissue pieces of method C need to be treated with collagenase. In addition, the main difference between method B and method C is that method B does not need to use a dissociator, and method C requires a dissociator.

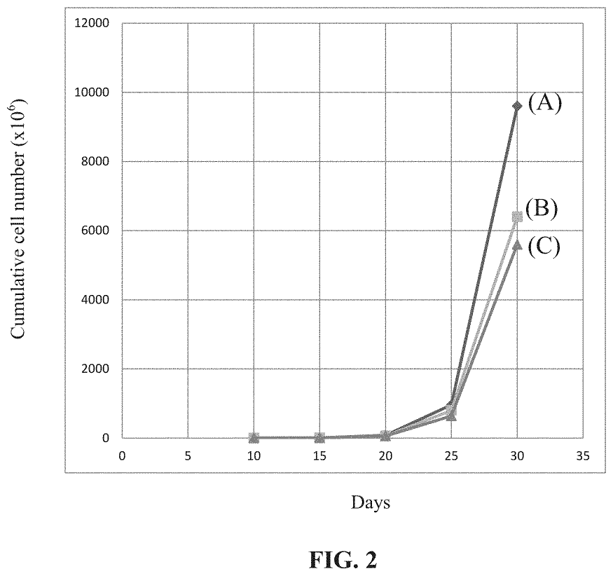

FIG. 2 shows the cumulative cell number of three different methods for hernia mesenchymal stem cell (MSC) cultivation. As a result, it was found that the method of the present invention (namely method A) can cultivate the largest number of mesenchymal stem cells, followed by method B and method C. In particular, the number of mesenchymal stem cells cultured by method A is about 1.46 times that of method B and 1.73 times that of method C, indicating that the mesenchymal stem cells cultured by the method of the present invention have high cell division ability, and the mesenchymal stem cells have high yield.

The mesenchymal stem cells isolated and purified by the method A of the present invention can be used for tissue repair. The tissue repair is to remove the hernia sac originally intended to be discarded during the operation, and the method of the present invention (without collagenase) can be used for tissue repair of the patient at the same time in the same operation, including the hernia repair operation, or other parts, including but not limited to prostate, bladder, gastric cardia, body scar tissue, etc. Because the hernia sac derived mesenchymal stem cells in the present invention do not need to be treated with enzymes, they can be directly applied to the human tissue. However, the conventional method with enzymes (method B or method C) may have infection concerns, so it needs more consideration to directly apply to the human tissues which requires repairing.

### Example 2

### Plastic Adherence Properties and Morphology of Cultured Hernia Sac-Derived Cells

The presence of MSCs was determined by using the following minimum criteria for defining multipotent MSCs according to The International Society for Cellular Therapy: (1) adherence to plastic, (2) specific surface antigen (Ag) expression, and (3) multipotent differentiation potential.

This example first assessed the morphology of the cells retrieved from hernia sacs obtained following inguinal hernia surgeries and their ability to adhere to plastics. MSCs were placed in plastic culture plates to observe their morphology and ability for plastic adherence when maintained in standard culture conditions using tissue culture flasks. The hematopoietic cells, mature adipocytes, etc, would not attach to the plastic plate in this condition.

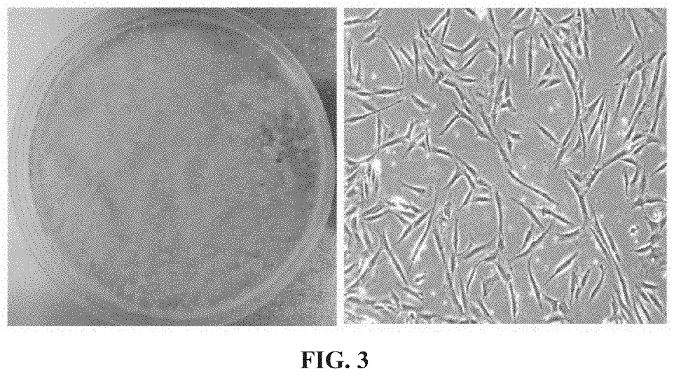

FIG. 3 shows plastic adherence properties and morphology of cultured hernia sac-derived cells. All cells from the 4 patients displayed a spindle-shaped morphology and exhibited adherence to plastics (see FIG. 3), partly indicating the presence of MSCs.

### Example 3

### Determining Reliable Positive Markers for Mesenchymal Stem Cell Identification

Positive markers selected for this example were based on existing literature (M. Dominici, K. Le Blanc, I. Mueller, et al., Minimal criteria for defining multipotent mesenchymal stromal cells. The International Society for Cellular Therapy position statement, Cytotherapy, 8 (4) (2006), pp. 315-317) and antibody availability for human MSCs. The markers CD105, CD73, and CD90 were used to assess expressions ≥95%, whereas markers CD45, CD34, CD11b, CD19, and HLA-DR were used to assess lack of expression (≤2%). All positive CD markers used (CD105, CD73, and CD90) had an expression of ≥95% when tested.

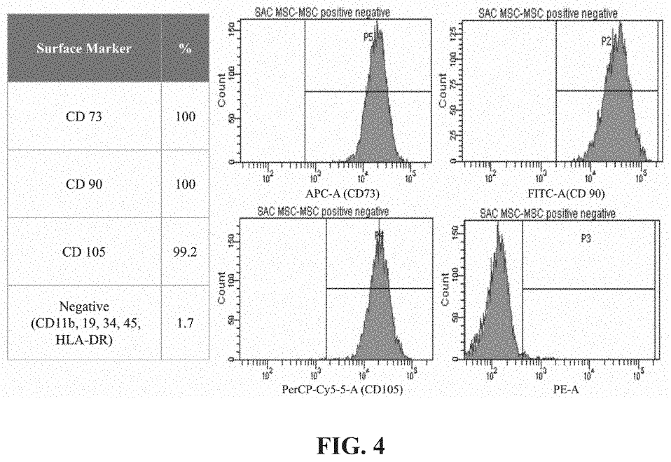

The cell surface immunophenotypic profile, along with the following positive CD markers, was determined: CD73 (100%), CD90 (100%), and CD105 (99.2%). The following negative CD markers were also assessed to further verify the presence of MSCs: CD11b, CD19, CD34, CD45, and HLA-DR (see FIG. 4). Accordingly, all negative CD markers tested (CD11b, CD19, CD34, CD45, and HLA-DR) had an expression of ≤2% (see FIG. 4), strongly suggesting the presence of MSCs.

### Example 4

### Differentiation Ability of Cells

When MSCs grew to 90% confluent, the medium was shifted to differentiation medium for the assay of trilineage capability. The differentiation media were StemMACS Osteodiff medium, StemMACS Chondrodiff medium, or StemMACS Adipodiff medium (Miltenyi Biotec), respectively, wherein the medium was shifted to StemMACS Osteodiff medium for von Kossa staining to verify osteogenesis; the medium was shifted to StemMACS Chondrodiff medium for Alcian blue stain to verify ability to undergo chondroblasts; the medium was shifted to StemMACS Adipodiff medium for Oil Red O staining to determine adipogenesis. After 3 weeks, cells underwent von Kossa staining to verify osteogenesis, Alcian blue stain to verify ability to undergo chondroblasts, and Oil Red O staining to determine adipogenesis.

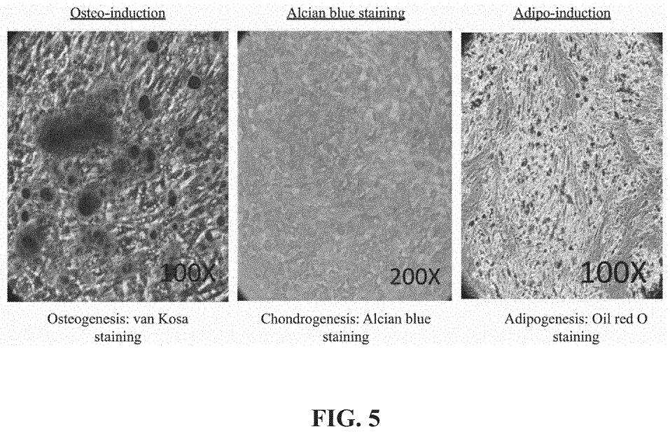

The in vitro differentiation of precursor cells into osteoblasts, adipocytes, and chondroblasts was determined through staining of in vitro cell cultures. Von Kossa staining confirmed differentiation into osteoblasts, Oil Red O staining demonstrated differentiation into adipocytes, and Alcian blue staining confirmed differentiation into chondroblasts. FIG. 5 revealed positive differentiation ability and identification of MSCs following von Kossa, Alcian blue, and Oil Red O staining. The uninduced cells showed no sign of differentiation.

### Example 5

Hernia Sac Derived Cells have More Mesenchymal Stem Cells than Subcutaneous Fat Derived Cells

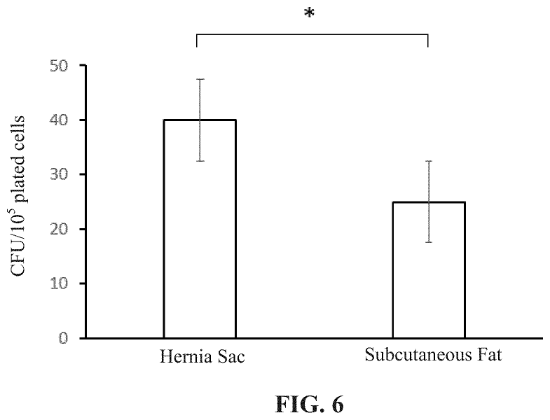

Because method B is the method used in general papers, and can quantitatively compare the number of cells treated in different tissues. Because the hernia sac tissue is richer in blood vessels, the obtained stromal vascular fraction (SVF) cells have significantly more colony forming units (CFUs) than the SVF cells treated with the same subcutaneous fat (see FIG. 6). This CFU is a cell population equivalent to mesenchymal stem cells. Therefore, from the results of this example, it can be seen that compared with fat tissue, more and healthy (with more number of cell divisions) mesenchymal stem cells can be obtained from the hernia sac tissue by the same method.

### Example 6

Hernia Sac Derived Mesenchymal Stem Cells have a Higher Number of Cell Divisions than Subcutaneous Fat Derived Mesenchymal Stem Cells

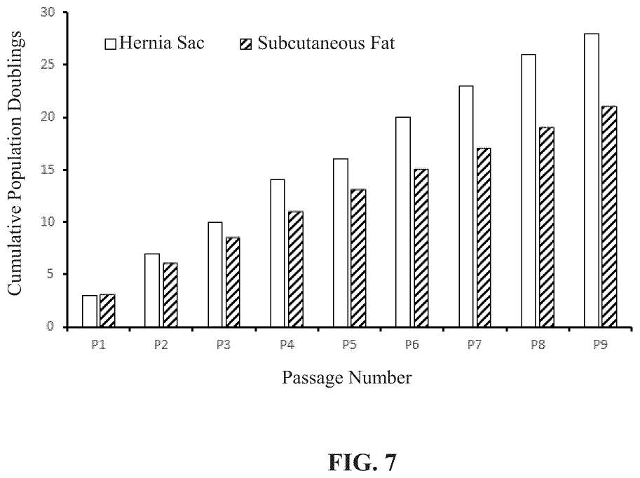

FIG. 7 shows that the hernia sac derived mesenchymal stem cells have a higher number of cell divisions and more accumulated cells than the subcutaneous fat derived mesenchymal stem cells. Therefore, the hernia sac derived mesenchymal stem cells purified and isolated according to the method of the present invention have high cell division ability.

In summary, the method for isolating and purifying mesenchymal stem cells from hernia sac and the method for tissue repair by using the mesenchymal stem cells from hernia sac of the present invention have the following effect. The mesenchymal stem cells of the present invention have high yield, high cell division ability, do not need to use enzymes as necessary reagents, and have high potential for application in regenerative medicine or tissue engineering for tissue repair (such as muscle tissue repair) that can be applied to the patient's own hernia repair defect structure, since many literatures prove that mesenchymal stem cells contribute to muscle tissue repair.

Although the present invention has been described with reference to the preferred embodiments, it will be apparent to those skilled in the art that a variety of modifications and changes in form and detail may be made without departing from the scope of the present invention defined by the appended claims.

What is claimed is:

1. A method for isolating and purifying mesenchymal stem cells from hernia sac, comprising the following steps:
(a) obtaining a hernia sac specimen and cutting the hernia sac specimen into tissue pieces;
(b) washing the tissue pieces and standing for a first predetermined time to form a supernatant and washed tissue pieces, then removing the supernatant;
(c) adding a first medium to the washed tissue pieces and performing a homogenization treatment, followed by a resuspension treatment to form a mixture comprising the mesenchymal stem cells, filtering the mixture comprising the mesenchymal stem cells to obtain a filtrate, and inoculating the filtrate into a flask for culturing for a second predetermined time; and
(d) separating the mesenchymal stem cells attached on the flask, then adding a second medium and continuing to culture for a third predetermined time, thereby purifying the mesenchymal stem cells;
wherein the step (b) is performed without the addition of an enzyme.

2. The method according to claim 1, wherein the tissue pieces have a volume of 1-2 mm³.

3. The method according to claim 1, wherein the first predetermined time is 4-6 minutes.

4. The method according to claim 1, wherein step (b) is repeated no more than twice.

5. The method according to claim 1, wherein both the first medium and the second medium are alpha-minimal essential medium.

6. The method according to claim 5, wherein the first medium or the second medium is further supplemented with 10% fetal bovine serum (FBS) and 1% solution comprising penicillin and streptomycin.

7. The method according to claim 1, wherein the second predetermined time is 24-48 hours.

8. The method according to claim 1, wherein in step (d), the mesenchymal stem cells attached on the flask are separated by trypsin.

9. The method according to claim 1, wherein the third predetermined time is 7-10 days.

10. The method according to claim 1, wherein the hernia sac is from an inguen.

11. A method for tissue repair, comprising administering to a subject in need thereof a pharmaceutical composition comprising an effective amount of mesenchymal stem cells from hernia sac, wherein the mesenchymal stem cells from hernia sac are isolated and purified by the method according to claim 1.

12. The method according to claim 11, wherein the tissue repair is muscle tissue repair.

13. The method according to claim 11, wherein the tissue pieces have a volume of 1-2 mm³.

14. The method according to claim 11, wherein the first predetermined time is 4-6 minutes.

15. The method according to claim 11, wherein step (b) is repeated no more than twice.

16. The method according to claim 11, wherein both the first medium and the second medium are alpha-minimal essential medium.

17. The method according to claim 16, wherein the first medium or the second medium is further supplemented with 10% fetal bovine serum (FBS) and 1% solution comprising penicillin and streptomycin.

18. The method according to claim 11, wherein the second predetermined time is 24-48 hours.

19. The method according to claim 11, wherein in step (d), the mesenchymal stem cells attached on the flask are separated by trypsin.

20. The method according to claim 11, wherein the third predetermined time is 7-10 days.
